(19) United States
(12) Patent Application Publication
(10) Pub. No.: US 2003/0089791 A1
(43) Pub. Date: May 15, 2003

(54) Vaporization indicator film

(76) Inventors: Yong S. Chen, Pleasanton, CA (US); Casper W. Chiang, Pleasanton, CA (US)

Correspondence Address: Ray K. Shahani, Esq., ATTORNEY AT LAW, Twin Oaks Office Plaza, 477 Ninth Avenue, Suite 112, San Mateo, CA, 94402-1854, US

(21) Appl. No.: 10/003,735

(22) Filed: Nov. 14, 2001

(51) Int. Cl. A61L 9/04

(52) U.S. Cl. 239/35

(57) ABSTRACT

A visual indicator microporous membrane that provides controlled-release of active ingredients, and prevents formulation leakage for use such as with an insecticidal or fragrance emanator device. During use, the semiporous membrane visually indicates transmission of active ingredient vapor to the consumer. These micropores that are normally opaque become saturated and turn from opaque to clear when in use, and when additional volatile material remains. Upon depletion of the volatile material from the reservoir and subsequently from the membrane, the membrane turns opaque again. In a preferred embodiment, the container comprises a protective shield at least partially covering the porous membrane.

### FIELD OF THE INVENTION

A microporous membrane that visually indicates the beginning, in progress, and point of depletion or end of use of liquid or vapor products, to also provide controlled-release of active ingredients and to prevent formulation leakage. During use, the semiporous membrane acts as a visual indicator to indicate transmission of active ingredient vapor, such as of an insecticide or fragrance emanator device. The micropores of the normally opaque membrane become saturated and thus the membrane film turns from opaque to clear during use. Upon depletion of the volatile material, the film reverts back to an opaque state.

### BACKGROUND OF THE INVENTION

For odorless vapor products such as insect repellent or insecticidal mosquito mats and emanators it is difficult, if not impossible, for consumers to know if ro when the product is being used or if it is completely consumed. Thus, there is a strong need for an indicator system to enable the consumer to conveniently and efficiently visually inspect and observe delivery and/or depletion of the active ingredients.

For such odorless vapor products it is also very difficult to control the evaporation rate. Quick evaporation rate decreases the life time of the product and forces the consumer to replace the product more frequently. Slow evaporation rates may compromise the effectiveness of the product.

There are many disadvantages with the technology currently known in the art. Mosquito emanators are currently being sold in unit-dose packages and are constructed with a metal tray and a plastic film as the lid. Within this tray is a formulation, usually a liquid or gel, containing the active ingredients. The consumer will put this metal tray into a mat heater (sold separately, but very common in Asian and Latin America countries) and the active ingredients contained in the metal tray will vaporize at elevated temperature. Depending on the vapor pressure, the active will permeate through the plastic film at certain rate and kill or repel the mosquitos inside a room of a typical household where the mat is applied. Typically a single package will last about 7 to 45 days of continuous use. The control of the permeation and maintaining a constant rate through out the usage cycle is critical. The selection of the plastic film depends on the permeation rate which in turn depends on the vapor pressure of the active ingredients.

The current art in the market uses clear polyester (PET) film with perforations (pinholes) or a polypropylene (PP) film without pinholes. One disadvantage of this approach is difficulty in controlling the size and number of the pinholes, therefore the vaporization or permeation rate of active ingredients is not easily controlled. If the pinholes are too big the active will be used up too soon (5-30 days vs. 745 days target).

Another disadvantage of the currently available technology is that the formulation will leak through the perforations. Since the formulation is either in liquid or gel state, if the holes are too big or too many, it will leak out of the package.

U.S. Pat. No. 4,512,933 issued Apr. 23, 1985 to Harden teaches apparatus for dispensing volatile substances. The apparatus provides for dispensing volatile substances which includes a housing with an oscillating piezoelectric element. This system further teaches an isotatic polypropylene film containing pores between about a micron and 0.02 micron. Diffusion is controlled by fusing portions of the membrane over portions of its area such as by heat sealing. This invention does not provide a solution to the problem that once activated or opened, the container will continue to dispense volatile material through its porous membrane.

U.S. Pat. No. 4,605,165, U.S. Pat. No. 4,614,299, and U.S. Pat. No. 4,387,849 teach a process for dispensing a volatile composition of matter from a container into the atmosphere surrounding the container at a controlled and steady rate. The apparatus includes a hollow totally enclosed structure comprising a thin shell totally enclosing an inner void, the thin shell being comprised of a thin polymer. The volatile composition of matter is placed in the hollow totally enclosed structure. The hollow container collapses when the ingredient is used up allowing the user to detect the exhaustion of the active ingredients. The disadvantage of this invention is the need for new formulation and increased production costs associated therewith.

U.S. Pat. No. 5,120,594 teaches a microporous polyolefin shaped article that comprises a polyolefin substrate of substantially skinless areas having high microporosity and skinned areas of reduced microporosity.

U.S. Pat. No. 5,993,954 teaches a temperature sensitive microporous film consisting of different layers having different melting points to control release of the active ingredients.

An alternative technique to provide controlled release of active ingredients is using an osmotic delivery system. U.S. Pat. No. 5,798,119 teaches an osmotic delivery device that provides for the controlled release of a beneficial agent to a non-aqueous environment with a semi-permeable hydrophobic microporous membrane.

Using a non-perforated film or an osmotic delivery system does not address the leakage issue. This approach requires new formulation of active ingredients or requires consumers to purchase new mat heaters due to different temperature requirements of the formulation.

Yet another disadvantage of the available technology is that the consumer is unable to tell if the active ingredient is being released and thus providing any protection against the mosquito. In addition, the consumer cannot tell if the formulation is used completely and needs replacement.

Various indicator systems are known to the art but they all have disadvantages that limit their use. One method uses a synchronizing fragrance to indicate that active ingredients are present and no fragrance when the formulation is depleted. This approach needs new development and will increase the overall cost. Furthermore, it does not address the leakage issue.

Another method is use of a colored formulation, which will change color when the active ingredients are used. This approach is taught by WO 00/69260, published on Nov. 23, 2000. The disadvantages is the color change is gradually visible and only at the end of the life can it be determined that the product is used up.

One class of indicators such as one taught in the U.S. Pat. No. 4,128,508 is based on the change in color of a PH indicator combined with a slowly evaporating acid or base. The disadvantage of such systems is possibility of leakage of dangerous acidic or basic medium which could harm the consumer and also the unpleasant smell of those ingredients.

U.S. Pat. No. 4,248,597 teaches a depletion indicator for removable substances based on a PH indicator. As the substance being delivered passes through a permeable membrane or porous substrate, a PH change occurs and the color of the PH indicator changes and indicates that the substance being delivered is exhausted.

Another life-time indicator of the prior art is described in U.S. Pat. No. 4,356,969 issued Nov. 2, 1982 to Obermayer et al. which teaches a vapor dispenser and method of use. In this system, color change upon evaporation acts as a lifetime indicator and makes the user aware that the vapor dispenser is depleted. In certain cases, the change in color is the transformations, after volatilization, of a mixture of dyes from green to an apparent white. Alternatively the color change can be achieved by a change in acidity, basicity or solvent character of the liquid as the more volatile components of the liquid evaporate and the change in the composition effects a color change in an indicating dye.

U.S. Pat. No. 4,824,707 teaches an air freshener unit having an impermeable backing sheet that is adherable to a substrate, and a facing sheet laminated to the backing sheet. Trapped between the two sheets is a supply of a volatile fragrance. The air freshener unit when activated exhibits an artwork which remains visible until the unit approaches exhaustion, at which point it fades out to signal the exhausted condition of the unit. This system is non-sealable, and once ruptured, the volatile fragrant material is exhausted completely.

U.S. Pat. No. 4,824,827 teaches an indicator composed of effective amount of a polar indicator dye. The disadvantage in this invention is that the ingredient component should vary to contain a proton donating compound that are not compatible with all active ingredients.

Yet another class of indicators as taught in the U.S. Pat. No. 4,921,636 is based on impregnating a porous material with a volatile liquid which becomes visually observable when the volatile liquid evaporates. This invention is directed to various ways to change the visual properties of a carrier material in the volatile composition. For instance, a transparent porous carrier material can be impregnated with a colored volatile liquid in a manner such that the carrier remains transparent but acquires a different color. Also, a clear carrier material can also be impregnated with a volatile material, such as a volatile liquid, which then turns the system cloudy, and upon volatilization of the active ingredients, the composition reverts back to clear, with or without an accompanying change in color.

U.S. Pat. No. 5,647,052 teaches a volatile substance dispenser which provides an indication of the dissipation of a quantity of volatile substance by changing an electrical signal level after a time duration corresponding to an expected period time for the quantity of volatile substance to disseminate.

As such, it can be seen that currently available technology has many shortcomings and there us a need for an indicator film that allows the controlled release of insecticidal active ingredients and prevents leakage out of the container while indicating the release of the active ingredients.

Another shortcoming of the prior art is that containers or delivery systems of the prior art are difficult to use over long periods of time. Once activated, the containers continue to emit or dispense volatile material until depleted, and there is no means for preventing such volatilization of material when the device is not in use. Basically, once activated, they must be used until depleted, and there is no effective mechanism for re-sealing the activated cartridge.

### OBJECTS AND ADVANTAGES

An object and advantage of the present invention is to use a semi-permeable membrane that can address the limitations of the current technology and provide a product that is superior in areas of performance, safety, economics, and indication.

Another object and advantage of the present invention is to provide an indicator system of indicating whether the active ingredients are being released.

Another object and advantage of the present invention is to provide a better control of the release of the active ingredients and therefore maintaining a constant permeation rate through out the product cycle.

Another object and advantage of the present invention is to provide a safer, non-leaking volatile material product container.

Yet another object and advantage of the present invention is its compatibility with current heaters or emanator devices in the market so that the consumer does not needs to buy new ones.

A further object and advantage of the present invention to prevents evaporation of the active ingredients during non-use of the reservoir portion in an insecticide emanator device.

It is yet a further advantage and objective of the present invention to provide a delivery system for volatile materials which can be activated to provide a visual indication of remaining volatile material, and which continues to provide this visual indication even not in use.

It is an object and advantage to provide a system which once activated, will be sealed when not in use to prevent evaporative loss over time and when not in use.

### SUMMARY OF THE INVENTION

The present invention is a visual indicator microporous membrane that provides controlled-release of active ingredients, and prevents formulation leakage for use such as with an insecticidal or fragrance emanator device. During use, the semiporous membrane visually indicates transmission of active ingredient vapor to the consumer. These micropores that are normally opaque become saturated and turn from opaque to clear when in use, and when additional volatile material remains. Upon depletion of the volatile material from the reservoir and subsequently from the membrane, the membrane turns opaque again. In a preferred embodiment, the container comprises a protective shield at least partially covering the porous membrane.

Another embodiment of the present invention incorporates vapor non-permeable film with a window of an opaque semi-permeable film, which will turn clear when the active ingredient is being vaporized and the film will turn back into opaque when the active ingredient is completely consumed.

In summary, in addition to control of delivery of volatile material by control and selection of pore size, the present invention controls delivery of volatile material by controlling the temperature and surface area. The prior art utilizes thick films, typically on the order of between about 2 mil and 400 mils, where the present invention is directed to membranes less than or equal to about 2 mils thickness. In the prior art, the concentration gradient across the film acts as a driving force. Thus, the delivery rate of transmission is a function of film thickness, whereas in the present invention, the temperature is the driving force. The temperature raises vapor pressure of volatile material to provide the desired delivery rate of transmission through the film. Thus, the delivery rate of the present invention is a function of temperature, “not the film thickness”.

### BRIEF DESCRIPTION OF THE DRAWINGS

The invention is illustrated below and represented schematically in the following drawings:

FIG. 1 is a representative schematic drawing of the container environment of the present invention.

FIG. 2 shows a schematic view of a preferred embodiment of a container 100 sealed with the indicator film 108 of the present invention.

FIG. 3 is a representative view of a heater device environment and apparatus 200 in which a container 100 with the membrane 108 described in the current invention is used.

FIG. 4A is a cross-sectional view of a preferred embodiment of the container 100 with the membrane 108 of the present invention prior to activation.

FIG. 4B is a cross-sectional view of a preferred embodiment of the container 100 with the membrane 108 of the present invention after activation.

FIG. 4C is a cross-sectional view of a preferred embodiment of the container 100 with the membrane 108 of the present invention after all the active ingredient has been used up.

FIG. 5 is a cross-sectional view of another preferred embodiment of the container 600 with the membrane 608 with protective shield member 610 of the present invention.

FIG. 6 is a representative graph of experimental data obtained during the investigation of a preferered embodiment of the apparatus and method the present invention.

### DETAILED DESCRIPTION OF THE PREFERRED EMBODIMENT

The description that follows is presented to enable one skilled in the art to make and use the present invention, and is provided in the context of a particular application and its requirements. Various modifications to the disclosed embodiments will be apparent to those skilled in the art, and the general principals discussed below may be applied to other embodiments and applications without departing from the scope and spirit of the invention. Therefore, the invention is not intended to be limited to the embodiments disclosed, but the invention is to be given the largest possible scope which is consistent with the principals and features described herein.

It will be understood that in the event parts of different embodiments have similar functions or uses, they may have been given similar or identical reference numerals and descriptions. It will be understood that such duplication of reference numerals is intended solely for efficiency and ease of understanding the present invention, and are not to be construed as limiting in any way, or as implying that the various embodiments themselves are identical.

Apparatus

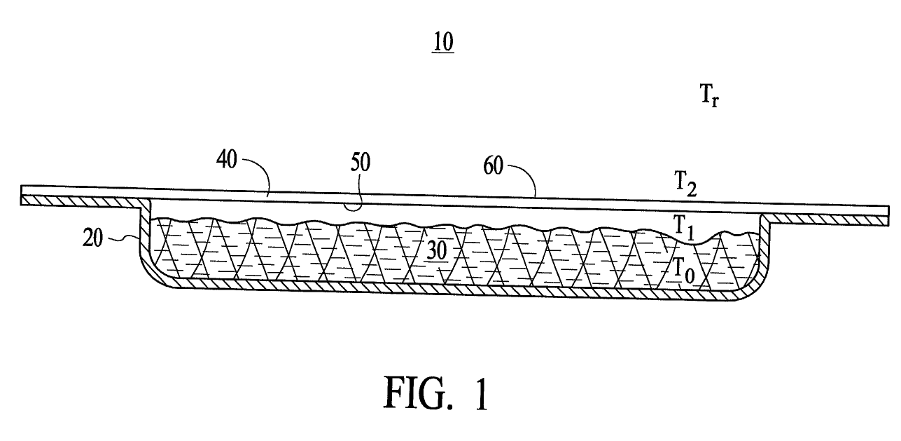

FIG. 1 is a representative schematic drawing of the container environment 10 of the present invention. It will be understood that the container apparatus 10 would typically involve a reservoir portion 20 to contain reserve supply of volatile material 30, such as insecticide, fragrance, other medicinal or therapeutic, etc. A “porous” membrane 40 is placed over the top portion of the reservoir 20 to contain the volatile material 30.

It will be understood that the temperature T₁ is room temperature in this representative system and T₀ is the temperature of the supply of the volatile material 30 remaining in the reservoir 20 of the container 10. before use or after use, T₀ will be essentially equivalent to T₁, i.e., T₁≈T₀. In use, i.e., when placed into a heating device, the container 10 will rise in themperature, and thus T₀ becomes greater.

The inside surface 50 of the membrane 40 is at temperature T₁ and the outside surface 60 of the membrane 40 is at temperatue T₂. Thus, T₂≈T₁. As vaporization occurs, the membrane 40 begins to heat up, and thus both T₁ and T₂ rise above T₁. If the membrane 40 is one of the prior art, there is a temperature gradient between the inside surface 50 of the membrane 40 at temperature T₁ and and the outer surface 60 at temperatue T₂, in other words, T₁>T₂. As described above, in this case the temperature gradient is the driving mechanism behind the diffusion process. However, in the present invention, the membrane 40 is a thin film, and T₀>>T₁≈T₂, and therefore the temperature is the determining factor in the diffusion process.

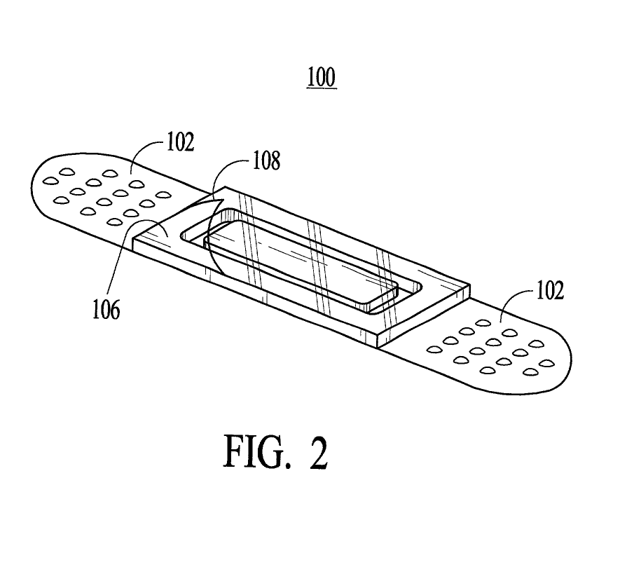

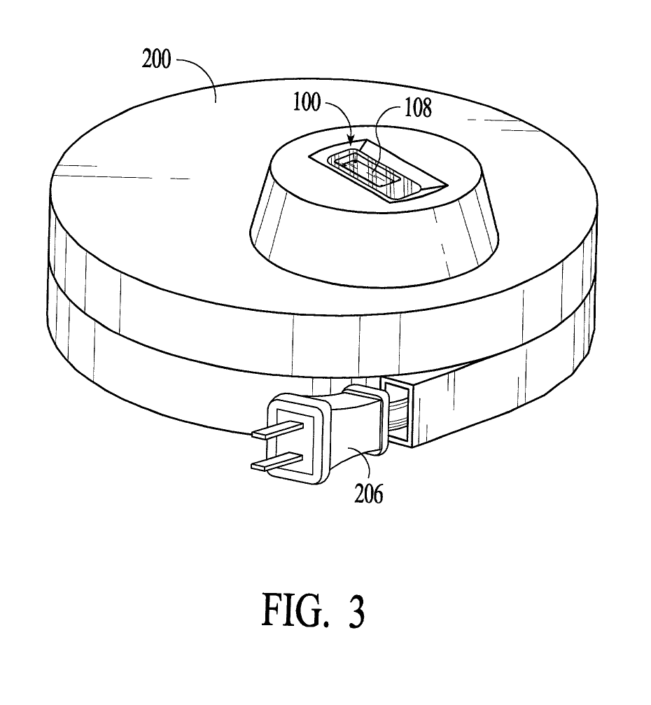

FIG. 2 shows a schematic view of a preferreed embodiment of the container 100 of the present invention. End tabs 102 are useful for inserting the container 100 into an emanator device (shown in FIG. 3). Indicator film 108 covers the entire opening or upper portion of the container 100. The device 100 has a reservoir 106 which contains the material to be volatilized 306, i.e., insecticidal composition, medicinal compounds or other therapeutic vapors, aromas, fragrances, etc.

In a preferred embodiment, indicator film 108 is a hydrophobic film that has 35-55% porosity and with pore size from 0.04×0.12 micron to 0.075×0.25 micron. The film can be heat sealed onto the metal tray. When the active ingredients is being vaporized and released through the membrane, the membrane becomes clear. The vapor of the active ingredients saturate the micro pores in the membrane and thus the film turns from opaque to clear. When the active is used up or leaves the micro pores of the membrane108, the film turns from clear to opaque.

The present invention utilizes the containers of the prior art for containing the insecticidal or other composition 306. In a preferred embodiment of the present invention, the containers 100 described and claimed in co-pending U.S. patent application Ser. No. 09/870,115 filed May 30, 2001, incorporated herein by reference in its entirety, are particularly adapted for such prolonged and delayed, controlled rates of delivery of insecticidal composition 306, medicinal compounds or other therapeutic vapors, aromas, fragrances, etc.

FIG. 3 is a representative view of a heater device apparatus 200 and environment in which a container 100 with the membrane 108 described in the current invention is used. An electrical connector 206 supplies power to an electrical resistor element (not shown) which causes evaporation of the active ingredient.

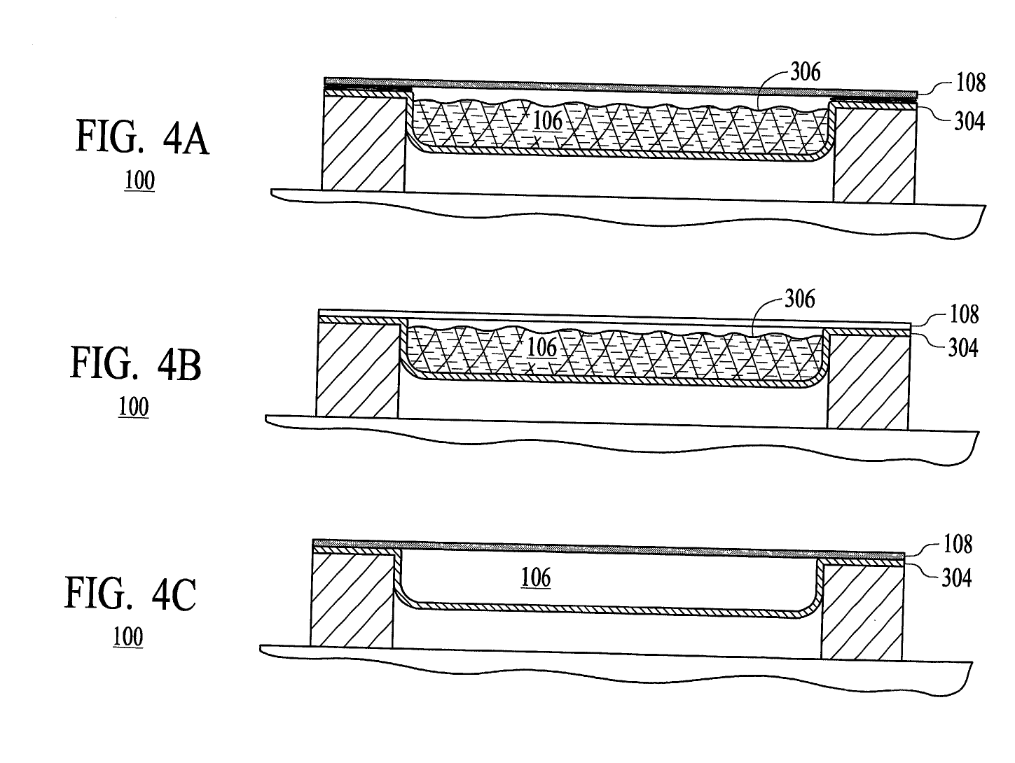

FIG. 4A is a cross-sectional view of a preferred embodiment of the container 100 with the membrane 108 of the present invention prior to activation. In this view, the indicator film 108 is opaque. The indicator film membrane 108 has not been activated, and the system has not been used yet. Activation can take place once the container 100 is heated for the first time. The indicator film 108 will turn from opaque to clear as the vapor of the active ingredients saturate the micro pores in the membrane108.

FIG. 4B is a cross-sectional view of a preferred embodiment of the container 100 with the membrane 108 of the present invention after activation. The indicator film 108 still seals the reservior 106 which holds the volatile compostion 306, but when the container is heated volatile material can be released.. The indicator film 108 is also still sealed to the container 100 at sealing surface 310. In this state, the membrane 108 is transparent, translucent or clear.

FIG. 4C is a cross-sectional view of a ptreferred embodiment of the container 100 with the membrane 108 of the present invention after all the active ingredient has been used up. The indicator film 108 has turned back from clear to opaque when the active is used up or leaves the micropores of the membrane 108, thus indicating the depletion of the volatile composition 306 (not shown).

In a preferred embodiment of the membrane 108 of the present invention, a semi-permeable polypropylene film is used. The trade name for this film is Celgard, manufactured by Celgard, division of Hoechst Celanese. Several grades of this film can be used: Celgard 2400, 2402, 2500, 2502, and 4560. These hydrophobic films have 35-55% porosity and with pore size from 0.04×0.12 micron to 0.075×0.25 micron. The film 108 can be heat sealed on to the metal tray or other portion of the container 100. When the active ingredients are being vaporized and released through the membrane 108, the membrane became clear. The vapor of the active ingredients saturate the micro pores in the membrane 108 and thus the film turns from opaque to clear. When the active is used up or leaves the micro pores of the membrane 108, the film 108 turns from clear to opaque.

Another embodiment of this invention is using other semi-permeable films 108 including Tyvek film made by DuPont. This is a non-woven film made of polyethylene fiber. The film 108 has greater vapor transmission rate with noticeable change in film 108 transparency. Tyvek film could serve as an on/off indicator 108 also but the change is not as dramatic as shown in Celgard film.

Another embodiment of this invention incorporates the semi-permeable membrane 108 within a non-permeable membrane. By varying the area of the semi-permeable film 108, the desirable release rate of active ingredients is achieved. The release of the actives can be better controlled by changing semi-permeable area size therefore maintaining a constant permeation rate through out the product cycle for different duration products.

The membrane 108 also prevents evaporation of the active ingredients during non-use of the reservoir portion in an insecticide emanator device 200. The prior art utilizes thick films, typically on the order of between about 2 mil and 400 mils, where the present invention is directed to membranes less than or equal to about 2 mils thickness.

The semi-permeable film 108 can be heat sealed onto the metal tray or other portion of the container 100. The non-permeable film is Aluminum/PP or metallized PE/PET film. These films can be heat sealed onto semi-permeable films 108.

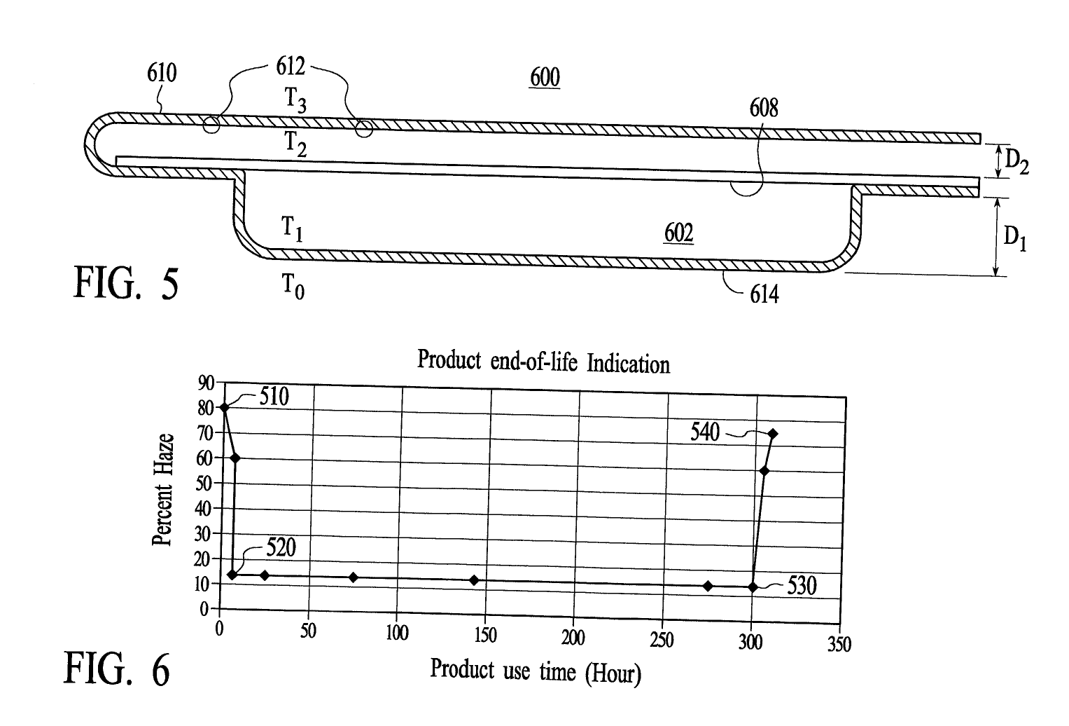

FIG. 5 is a cross-sectional view of another preferred embodiment of the container 600 with the membrane 608 of the present invention with a protective shield member 610 of the present invention. In a preferred embodiment, the container 600 has a reservoir portion 602 which would contain the volatile material to be dispensed or diffused (not shown). The small-pore thin-film membrane 608 is directly adjacent the reservoir portion 602. In this embodiment, there is a protective shield portion 610 covering the membrane 608. Small holes 612 or pores are positioned within the upper surface of the protective shield 610 to allow diffusion or transport of volatile material during use. It will be understood that this protective shield 610 is useful for controlling the temperature T₂ of the membrane 608.

It will also be understood that by establishment of the protective shield 610 shown in FIG. 5, there is created additional geometry which may have an impact on performance thereof. In particular, while the membrane 608 may be a given distance D₁ raised above the lower surface 614 of the container 600, the protective shielf 610 is raised a distance D₂ over the membrane 608. Furthermore, while the heater device (not shown) may be set at a temperature T₀ and the reservoir is maintained at a temperature T₁, the thin-film membrane 608 will be at temperature T₂, but the protective shield 610 may be at a lower temperature, closer to room temperature Tᵣ. Therefore, it is apparent that establishing this temperature gradient, where T₀ is greater than or equal to about T₁. T₁ may be slightly higher than or equal to T₂, the temperature of the porous membrane 608. Finally, T₃ may be slightly higher or equal to T₂, the temperature of the protective shield 610.

Insecticidal and Other Compositions

The volatile material 306 or insecticide be any one or combination of insecticides and insect repellents, and/or other active agents. Particularly preferred are organic phosphorous insecticides, lipidamide insecticides, natural repellents as citronella oil, natural pyrethrins or pyrethrum extract, and synthetic pyrethroids. Suitable synthetic pyrethroids are allethrin as Pynamin, d-allethrin as Pynamin forte, benfluthrin, bifenthrin, bioallethrin, S-bioallethrin, esbiotrin, esbiol, bioresmethrin, cycloprothrin, cyfluthrin, beta-cypermethrin, cyphenothrin, deltamethrin, empenthrin, esfenvalerate, fenpropathrin, fenvalerate, flucythrinate, tau-fluvalinate, kadethrin, permethrin, phenothrin, prallethrin as ETOC, resmethrin, tefluthrin, tetramethrin, transfluthrin, or tralomethrin.

Fragrances 306 and/or deodorizers, such as a terpene based deodorizer fragrance may also be used in the reservoir portion 106 of the container 100 of the present invention. Further, volatile fragrances, disinfectants, or other air quality modifying agents may be used, such as glycols, trimethylene, and dipropylene. In addition, organic acids that are compatible with the use of the substrate and the atmosphere can also be utilized.

The following table Table 1 is a listing of the vapor pressures of some of the compounds useful as active ingredients, carriers or solvents in the present invention, as well as a few compounds of the prior art with vapor pressures outside of the scope of this invention, shown for comparative purposes:

TABLE 1

| Componud | Vapor pressure, torr |
|---|---|
| Allethrin | 1.2 × 10^{-4 (30}° C.) |
| Bioresmethrin | 3.34 × 10⁻⁴ (20° C.) |
| Deltamethrin | 1 × 10⁻⁷ (25° C.) |
| Bioallethrin | 3.3 × 10⁻⁴ (25° C.) |
| Prallethrin | 1 × 10⁻⁷ (23° C.) |
| Transfluthrin | 3.0 × 10⁻⁶ (20° C.) |
| Tefluthrin | 6.0 × 10⁻⁵ (20° C.) |
| Permethrin | 3.4 × 10⁻⁷ (25° C.) |
| Phenothrin | 1.4 × 10⁻⁷ (21.4° C.) |
| Hexanol (prior art) | 1 (24.7° C.) |
| Diethylene glycol monoethyl ether (prior art) | 8 × 10⁻² (20° C.) |
| Heptanol (prior art) | 5 × 10⁻¹ (20° C.) |
| Octanol (prior art) | 1.4 × 10⁻¹ (25° C.) |

It will be understood that the active ingredients or other compounds and compositions of the prior art have all been volatile at room temperature. In the present invention, the preferred embodiment contains a volatile material with a vapor pressure in the range of from about 10⁻⁴ Torr at room temperature to about 10⁻⁷ Torr at room temperature. In another preferred embodiment, the volatile material has a vapor pressure in the range of from about 10⁻⁴ Torr at room temperature to about 10⁻⁶ Torr at room temperature.

Other insecticidal and pesticidal compositions useful and within the scope of the present invention are described in co-pending U.S. patent application Ser. No. 09/207,397 filed Dec. 8, 1998, U.S. patent application Ser. No. 09/666,716 filed Sep. 20, 2000, and U.S. patent application Ser. No. 09/870,117 filed May 30, 2001, all hereby incorporated herein by reference in their entireties.

Preferred Methods of Use

It will be understood that the membrane film 108 for the container 100 of the present invention operates according to various mechanisms. In a preferred embodiment, the film 108 is opaque and is effective for sealing the reservoir 106 from preventing leakage of the volatile material when not desired or not in use. When in use, i.e., when the membrane is in use on a container device 100 such as for containing a fragrance, deodorizer or insecticides and the like and the container is placed into a heater device 200 as shown and known in the prior art, the membrane film 108 turns from opaque to clear, as a result of saturation of the membrane with the vaporized materials. In a preferred embodiment, it is one or more chemical components of the composition of the fluid or paste or vapor disposed and contained within the reservoir 106. In additional preferred embodiments, a color change is produced due to physical or chemical reaction of the volatile material with a component of the membrane or film 108 covering the reservoir 106. In such embodiments, the membrane 108 turns from opaque when not in use to clear when in use, or from one color when in use to another color when not in use or when in an alternate mode of operation.

It will be appreciated that, in distinction with the prior art, once the opaque membrane film of the present invention is activated, it becomes clear or transparent. Thereafter, it will remain in this clear transparent state until the volatile material to be realeased is depleted. It will also be noted as a distinct and novel aspect of the present invention that while the container filled with the volatile material such as insecticide can be removed from the heater device, in which case the delivery of volatile material is terminated. However, as stated above, the film remains transparent clear until the reservoir is completely and totally depleted.

Test Protocol and Results

The following is a description of exemplary tests conducted and data obtained therefrom. Clarity or optical transmission measurement of the membrane or thin film of prior art and of the present invention were taken to determine the efficacy of the present invention. These tests were conducted under conditions specified by and similar to American Standards and Testing Methods test method known as ASTM method D1003. A slight difference in instrument geometry was made. Haze (percentage) is calculated as ratio of Y diffuse transmission and Y total transmission.

The following data in the table Table 2 below was obtained using the methods and materials described herein:

| Sample ID | Haze |
|---|---|
| Polyethylene clear film with thickness of 1 mil | 7.88 |
| Celgard polypropylene film before use - 1 mil thickness | 86.90 |
| Celgard polyproylene film during use - 1 mil thickness | 21.37 |

FIG. 6 is a representative graph 500 of experimental data obtained during the investigation of a preferered embodiment of the apparatus and method the present invention. The graph 500 is a representation of the product end of life indication function of the present invention. Expressed in terms of the percent haze on the x-axis and product use time in hours on the y-axis, the graph 500 shows the initial clearing of the opaque small-pore thin-film membrane from the point 510 at which it exists in the initial opaque state where it is inactivated and unloaded, until it reaches a point 520 of significant transparency. The film remains transparent through the greatest majority of the entire product use time, until a point 530 at which the volatile material in the reservoir is essentially exhausted. The opacity of the film membrane rises rapidly and the transparency is essentially gone at point 540. During the comparatively short period of time between point 530 and point 540, the reservoir is essentially already depleted or exhausted, and the only remaining diffusion is as a result of the transfer of individual molecules of volatile material formerly residing within pores in the membrane to a point outside of the membrane.

It will be important to re-emphasize that one aspect of the present invention is the distinction between “product use time” and “total time”. Therefore, while the graph 500 shown in FIG. 5 is demonstrative of such system of the present invention which has a product use lifetime of about 300 hours, it will be understood that those 300 hours could be spread out over 25 continuous days of use or 300 days of use not more than about 1 hour per day to longer, depending upon total usage. For example, a system of the present invention rated to operate for 30 days consecutively for 10 hour intervals would be advantageous and effective for home use by all those in areas where mosquitoes or other insects, or unpleasant smells and aromas, etc., are a nuisance. In comparison to the prior art, the present invention provides constant visualization throughout the useful lifetime of the system, i.e., the period of time between points 520 and 530, and the system can be used as little or as much as desired with essentially no increased hazing or diminution of clarity during that time period. Thus, the visual indicator is a small-pore film which becomes opaque when there is essentially no effective remaining amount of volatile material in the system.

Furthermore, the present system is a non-contact film, it does not require contact with a liquid form of the volatile material. Therefore, it can also be considered a form of clarifying membrane in the presence of any reserve or supply of volatile material. Additionally, the optical properties of the film or membrane of the present invention operate within the visual range of the electromagnetic radiation spectrum of any of those having wavelengths between about 400 nannometers and about 700 nannometers.

Unless defined otherwise, all technical and scientific terms used herein have the same meaning as commonly understood by one of ordinary skill in the art to which this invention belongs. Although any methods and materials similar or equivalent to those described can be used in the practice or testing of the present invention, the preferred methods and materials are now described. All publications and patent documents referenced in this application are incorporated herein by reference.

While the principles of the invention have been made clear in illustrative embodiments, there will be immediately obvious to those skilled in the art many modifications of structure, arrangement, proportions, the elements, materials, and components used in the practice of the invention, and otherwise, which are particularly adapted to specific environments and operative requirements without departing from those principles. The appended claims are intended to cover and embrace any and all such modifications, with the limits only of the true purview, spirit and scope of the invention.

We claim:

1. A system for indication of controlled release and depletion of vaporized volatile ingredients at temperatures above room temperature, the system comprising a microporous membrane sealed to a container containing ingredients to be vaporized, the membrane made of a material which when activated through the use of a heating device turns from opaque to essentially transparent or translucent during vaporization of volatile ingredients, the membrane remaining essentially transparent or translucent as long as there are ingredients to be vaporized remaining in the container and the membrane returning to opaque upon depletion of the ingredients to be vaporized, whereby the visual difference between the opaque and the essentially transparent or translucent microporous membrane serves as a depletion indicator for the system.

2. The system of claim 1, wherein the membrane is a polypropylene film having pore size of about 0.01 by about 0.1 micron to about 0.01 by about 1.0 micron, the film having an overall thickness of less than about 2 mils.

3. The system of claim 1, wherein the membrane film is less than about 2 mil in thickness.

4. The system of claim 1, wherein the volatile ingredients are one or more compounds selected from the group consisting of pesticides and insecticides, insect repellents, fragrances, air-fresheners and deodorizers.

5. The system of claim 1, wherein the volatile ingredients have a vapor pressure of about 10⁻⁴ Torr to about 10⁻⁷ Torr at about room temperature.

6. The system of claim 1, wherein the volatile ingredients are in a gel or solid form.

7. The system of claim 1, further comprising a temperature controller. whereby delivery of volatile material is controlled thereby.

8. A container for dispensing volatile materials into an atmosphere at temperatures above room temperature, the container adapted for use in a emanator device such as for insecticide, deodorizer or fragrance or the like, the container comprising:
a reservoir portion for containing volatile material to be dispensed;
a microporous membrane covering the reservoir for preventing the evaporation of the active volatile materials during non-use of the emanator device.

9. The container of claim 8, further comprising means for securing the membrane to the reservoir portion.

10. The container of claim 9, in which the securing means comprises a heat weld.

11. The container of claim 9, in which the securing means comprises an adhesive material.

12. The container of claim 8, further comprising a volatile material.

13. The container of claim 8, further comprising a protective shield member at least partially covering the porous membrane.

14. The container of claim 8, particularly adapted for dispensing volatile material which has a vapor pressure of about 10⁻⁴ Torr to about 10⁻⁷ Torr at about room temperature.

15. A semiporous membrane-type, depletion indicator film for controlled relase of volatile materials for use in an emanator device such as for insecticide, deodorizer or fragrance or the like, the film having a predetermined porosity for preventing undesired leakage of the volatile material and for allowing controlled release of vaporized volatile materials, the film further having an opaque visual appearance when not in use and the film having an essentially transparent appearance when releasing the volatile materials, whereby the essentially transparent appearance of the film indicates release of volatile material.

16. The depletion indicator film of claim 15 in which the membrane is microporus and in which the transparent appearance of the film during release of the volatile material is due to saturation of the microporous membrane by the volatile materials.

17. The depletion indicator film of claim 15 in which the membrane is microporus and in which the transparent appearance of the film during release of the volatile material is the result of a physical reaction between the the volatile material and the membrane film.

18. A dispensor of volatile materials adapted for use in a heater-type emanator device such as for insecticides, fragrances, deodorizer and the like, the dispensor comprising:
a reservoir portion for containing volatile material to be dispensed;
a small-pore membrane covering the reservoir for preventing the evaporation of the active volatile materials during non-use of the emanator device, the membrane also composed of a small pore size material which when saturated with the volatile material turns from an opaque film to an essentially transparent or otherwise translucent film, once activated by initial saturation of the membrane the film remains in the essentially transparent or otherwise translucent state until there is essentially no remaining volatile material in the reservoir.

19. The dispensor of claim 18, further comprising a protective shield member mounted to the dispensor and disposed essentially over at least a portion of the membrane covering the reservoir.

20. The dispensor of claim 19 in which the protective shield member is provided with at least one relatively small hole herein to provide additional control of release of the volatile material.

21. The dispensor of claim 19 in which the protective shield member is provided with a plurality of relatively small holes herein to provide additional control of release of the volatile material.

22. A method for determining depletion of a container for dispensing volatile materials, the container having a semiporous membrane mounted thereon for controlled release of volatilized materials, the method consisting of the following steps:
Activating the membrane of a new container, thereby converting the membrane from opaque to essentially transparent or translucent;
Using the container for dispensing volatile materials as usual; and
Observing the container at the point at which the semiporous membrane turns back to opaque, thereby indicating the container is essentially empty, or the end of life of the container.

23. The method of claim 22, further comprising the following step:
Discarding the essentially empty container and replacing with a new container.
